(19) United States
(12) Patent Application Publication
(10) Pub. No.: US 2024/0052350 A1
(43) Pub. Date: Feb. 15, 2024

(54) OLIGONUCLEOTIDE AND USE THEREOF AGAINST HEPATITIS B VIRUS AND HEPATITIS D VIRUS

(71) Applicant: ZHEJIANG PALO ALTO PHARMACEUTICALS, INC, Quzhou (CN)

(72) Inventors: Zhijian XI, Quzhou (CN); Zhongshan WU, Quzhou (CN); Chunping LU, Quzhou (CN)

(21) Appl. No.: 18/317,177

(22) Filed: May 15, 2023

Related U.S. Application Data
(63) Continuation of application No. PCT/CN2021/130757, filed on Nov. 15, 2021.

(30) Foreign Application Priority Data
Nov. 16, 2020 (CN) 202011282529.9

(51) Int. Cl. C12N 15/113

(52) U.S. Cl. CPC C12N 15/1131; C12N 2310/11; C12N 2310/17; C12N 2310/321; C12N 2310/3341; C12N 2310/315; C12N 2310/341

(57) ABSTRACT

Provided are an oligonucleotide and the use thereof against the hepatitis B virus and the hepatitis D virus. Specifically, provided is a compound, or a pharmaceutically acceptable salt, a hydrate or a solvate thereof, wherein the compound is a modified or unmodified oligonucleotide and has a length of 24-40 nt. The oligonucleotide has a core sequence as shown in SEQ ID NO.1: GTGCAGAGGTGAAX1X2X3AAGTGCAC (SEQ ID NO.1), wherein X1X2X3 is GCG, CCG or CCT; and each T in the core sequence may be independently substituted with U.

### CROSS-REFERENCE TO RELATED APPLICATIONS

This application is a continuation of International Application No. PCT/CN2021/130757 with a filling date of Nov. 15, 2021, designating the United states, now pending, and further claims to the benefit of priority from Chinese Application No. 202011282529.9 with a filing date of Nov. 16, 2020. The content of the aforementioned applications, including any intervening amendments thereto, are incorporated herein by reference.

### SEQUENCE LISTING INFORMATION

This application file contains a Sequence Listing submitted in computer readable ASCII text format (file name: sequence list.txt, date created: May 13, 2023, size: 30,739 bytes). The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The content of the Sequence Listing file is incorporated herein by reference in its entirety.

### FIELD OF THE INVENTION

The invention belongs to the field of biomedicine, and particulary relates to an antisense oligonucleotide and the use thereof against the hepatitis B virus and the hepatitis D virus.

### BACKGROUND OF THE INVENTION

Hepatitis B is a viral disease caused by hepatitis B virus (HBV) infection and its main routes of transmission include blood transmission, sexual transmission and mother-to-child transmission. According to the estimate of World Health Organization (WHO), more than 200 million people are chronically infected with HBV and 887,000 people die from complications of HBV infection in 2015 across the world. Among the adults infected with HBV, 90% of them are able to heal themselves, but 90% of infants infected with HBV will develop into chronic hepatitis. Chronic HBV infection may lead to liver fibrosis and further development to cirrhosis and hepatocellular carcinoma (HCC). In addition, some studies have shown that hepatitis B increases the risk of pancreatic cancer.

Hepatitis D virus (HDV) is a satellite virus of HBV, which relies on hepatitis B surface antigen (HBsAg) to form its complete infectious HDV virus particles. HDV infection can only occur in patients with HBV infection. The complications of HDV/HBV co-infection are obvious and significantly increase the rate of progression from liver fibrosis to cirrhosis. Currently, for patients with chronic HDV infection, only interferon therapy is adopted as an intervention method, and there is no marketed drug directly targeting on HDV virus. The existing therapeutic methods are not effective and have remarkable side effects.

HBV adheres to the surface of liver cells through low-affinity receptors, and then enters the liver cells through endocytosis with the help of specific receptors on the liver cell membrane. The nucleocapsid disassembles and imports rcDNA into the host cell nucleus. In the nucleus, rcDNA is converted into covalently closed circular DNA (cccDNA) through the DNA repair mechanism of cells. The cccDNA is the storage form of HBV genetic material in cells, and also the main transcription template of HBV. Host cells recognize the promoter and enhancer on cccDNA and transcribe 3.5 kb, 2.4 kb, 2.1 kb and 0.7 kb mRNA, of which 3.5 kb mRNA is pre-genomic RNA (pgRNA). The mRNA enters the cytoplasm and is translated into viral proteins, including core antigen (HBcAg), e antigen (HBeAg), surface antigen (HBsAg), x protein (HBx) and polymerase. Under the action of Polymerase, the negative sense strand of HBV DNA is synthesized by reverse transcription using pgRNA as a template, and then part of the sense strand is further synthesized using the negative sense strand as a template to form rcDNA. Meanwhile, HBcAg is assembled into a nucleocapsid to wrap rcDNA and form a viral core particle. The synthesized HBsAg is multimerized in the endoplasmic reticulum and transported to the Golgi apparatus to package the viral core particles, and the assembled viral particles are finally secreted out of the cell by budding.

After HBV infects human hepatocytes, two different particles are mainly produced: Dane particles and subviral particles (SVPs). The Dane particle is the complete HBV virus itself, including the viral nucleocapsid assembled from hepatitis B core antigen (HBcAg) and viral nucleic acid (RcDNA), and having a viral envelope composed of hepatitis B surface antigen (HBsAg); the subviral particle is a non-infectious particle composed of lipids, cholesterol, cholesteryl ester, and hepatitis B surface antigen (HBsAg). The HBsAg contained in SVP accounts for the vast majority (>99.9%) of the HBsAg in the patient's blood. HBV-infected liver cells also secrete an e-antigen (HBeAg) into the blood. Hepatitis B surface antigen (HBsAg), hepatitis B surface antibody (HBsAb), hepatitis B core antibody (HBcAb), hepatitis B e antigen (HBeAg) and hepatitis B e antibody (HBeAb) are important molecular markers for evaluating the drug intervention on the virus.

A large amount of hepatitis B surface antigen (HBsAg) in the form of subviral particles (SVP) in the blood of patients with chronic HBV infection can neutralize the specific hepatitis B surface antibody (HBsAb) secreted by B lymphocytes, leading to immune tolerance; but only a small number of HBV virus particles can escape the immune check, which may be one of the important reasons for HBV to maintain chronic infection. The HBsAg seroconversion (clearance of HBsAg from the blood and appearance of free HBsAb) is a recognized prognostic indicator of functional of viral infection after treatment. Another key reason for HBV maintaining the characteristics of chronic infection is that it synthesizes a stable circular DNA storehouse, i.e., HBV covalently closed circular DNA (cccDNA), in the nucleus of infected hepatocytes with the help of host DNA repair enzymes. The cccDNA can exist stably in hepatocytes for a long time and can be supplemented continuously. It can produce the nucleic acid RcDNA of HBV virus and the mRNA required for encoding all viral antigens through transcription and reverse transcription. Transcriptional repression or clearance of cccDNA is critical for curing or functionally curing HBV infection. Long-term treatment with nucleoside (nucleotide) analogues cannot completely eliminate cccDNA, nor can it inhibit its transcription; therefore, the expression level of hepatitis B surface antigen (HBsAg) is hardly affected by nucleoside (nucleotide) drugs. Immunomodulation can mediate humoral and cellular immunity, thereby inhibiting cccDNA transcription or clearing infected cells; however, a large antigen load can greatly inhibit the immune process, and greatly reduce the antigen, especially hepatitis B surface antigen (HBsAg). The immunomodulation is an effective means to help patients achieve durable immune control.

The drugs clinically used in the treatment of hepatitis B mainly include interferons and nucleoside (nucleotide) drugs. Interferon drugs include common interferon and pegylated interferon, while the latter includes Pegasys (PEG-IFNα-2a) and Peglntron (PEG-IFNα-2b). Nucleoside (nucleotide) drugs include lamivudine, telbivudine, adefovir dipivoxil, tenofovir disoproxil fumarate (TDF), tenofovir alafenamide fumarate (TAF), Entecavir, etc. These nucleoside drugs can effectively control virus replication and improve liver function, so they are widely used. Interferon needs to be administered by injection, and the individual reactions differ greatly, with obvious adverse reactions and poor curative effect. Nucleoside drugs only act on the replication process of the virus from pgRNA to rcDNA, and have no inhibitory effect on other links in the life cycle of hepatitis B virus. After long-term treatment, the negative conversion rate of hepatitis B e antigen (HBeAg) is still low, and very few patients can be negative for hepatitis B surface antigen (HBsAg). After patients were treated with entecavir (354 cases) and tenofovir (176 cases) for 48 weeks, the negative conversion rates of hepatitis B surface antigen (HBsAg) in patients with positive hepatitis B e antigen (HBeAg) were 2% and 3.2%, respectively; and the negative conversion rates of hepatitis B surface antigen (HBsAg) in patients with negative hepatitis B e antigen (HBeAg) were 0.3% and 0%, respectively. Since the existing treatment regimens cannot cure hepatitis B, patients need to take drugs for a long time, which may lead major side effects to patients. For example, long-term use of adefovir dipivoxil and tenofovir disoproxil fumarate can lead to nephrotoxicity and bone toxicity. Existing drug therapy or combination therapy fails to induce an effective immune response or HBsAg seroconversion that would provide durable control of the infection or a functional cure, except in a small part of patients (<3%). A cure or functional cure for chronic HBV infection is a huge unmet clinical need.

In summary, it is urgent to discover and develop new antiviral treatments in this field, particularly, a new therapy that can effectively inhibit hepatitis B virus antigens (HBsAg and/or HBeAg) and increase their seroconversion rates.

### SUMMARY OF THE INVENTION

An object of the present invention is to provide a new compound for the treatment of the hepatitis B virus/hepatitis D virus.

In a first aspect of the present invention, a compound, a pharmaceutically acceptable salt, a hydrate or a solvate thereof are provided, wherein, the compound is a modified or unmodified oligonucleotide, and the oligonucleotide has a length of 24-40 nt, preferably 26-38 nt, more preferably 30-36 nt;
and the oligonucleotide has a core sequence as shown in SEQ ID NO.1:

(SEQ ID NO. 1)
GTGCAGAGGTGAAX1X2X3AAGTGCAC

wherein, X₁X₂X₃ is GCG, CCG or CCT; and each T in the core sequence may be independently substituted with U;
wherein, the modification is one or more modifications selected from the following group:
(i) nucleoside modification; the nucleoside modification comprises 2′-O-methylated glycosyl modification, 2′-O-methoxyethylated glycosyl modification, and/or methylation modification of the C-5 position of cytosine;
(ii) modification of internucleoside bonds; the modification of internucleoside bonds is that part or all of internucleoside bonds in the oligonucleotide is substituted with a phosphorothioate internucleoside bond and/or a phosphorodithioate internucleoside bond.

In another preferred embodiment, X₁X₂X₃ is GCG.

In another preferred embodiment, the oligonucleotide has a structure represented by the formula I:

Z1-Z2-Z3 (I)
in the formula,
Z1 is a left extension sequence located at the 5′ end of the core sequence, and the left extension sequence has a length L1 of 0-10 nt; and when L1≥1, the left extension sequence comprises nucleotides from position 11-L1 to position 10 in 5′-TCCATGCGAC-3′ successively (i.e., when L1=1, Z1 is C; when L1=2, Z1 is AC; . . . ; when L1=10, Z1 is 5′-TCCATGCGAC-3′);
Z2 is the core sequence;
Z3 is a right extension sequence located at the 3′ end of the core sequence, and the right extension sequence has a length L2 of 0-12 nt, and when L2>1, the right extension sequence comprises nucleotides from position 1 to position L2 in 5′-ACGGTCCGGCAG-3′ successively;
and each T in the oligonucleotide may be independently substituted with U.

In another preferred embodiment, L1 is 0, 1, 2, 3, 4, 5, 6, 7, 8, 9, or 10.

In another preferred embodiment, L2 is 0, 1, 2, 3, 4, 5, 6, 7, 8, 9, 10, 11, or 12.

In another preferred embodiment, L1+L2 equals 1, 2, 3, 4, 5, 6, 7, 8, 9, 10, 11, 12, 13, 14, 15, or 16.

In another preferred embodiment, all internucleoside bonds in the oligonucleotide are substituted with phosphorothioate internucleoside bonds.

In another preferred embodiment, the oligonucleotide has one or more nucleotides with nucleoside modifications.

In another preferred embodiment, the oligonucleotide has nucleoside modifications in a region selected from the group consisting of 2-6 nts at the 5′ end, 2-3 nts in X₁X₂X₃, 2-6 nts at the 3′ end, or a combination thereof.

In another preferred embodiment, the 2-6 nts at the 5′ end refers to 2-6 consecutive nucleotides from the 5′ end.

In another preferred embodiment, the 2-6 nts at the 3′ end refers to 2-6 consecutive nucleotides from the 3′ end.

In another preferred embodiment, the 2-3 nts in X1X2X3 refer to 2-3 consecutive nucleotides therein.

In another preferred embodiment, the oligonucleotide is also optionally modified at 1, 2 or 3 extending from the 3′ end and/or 5′ end of X₁X₂X₃, and these modifications are consecutive in 2-3 modified nts in X₁X₂X₃.

In another preferred embodiment, the modifications at 1, 2 or 3 extending from the 3′ end and/or 5′ end of X₁X₂X₃ and the 2-3 modified nts in X₁X₂X₃ are consecutive.

In another preferred embodiment, the oligonucleotide has nucleoside modifications in 2-6 nts at the 5′ end, 2-3 nts in X₁X₂X₃, and 2-6 nts at the 3′ end.

In another preferred embodiment, there is no nucleoside modification in the gap region Gap1 between the 2-6 nucleoside-modified regions at the 5′ end and X₁X₂X₃, or there is part or all of nucleoside modifications.

In another preferred embodiment, there is no nucleoside modification in the gap region Gap 2 between X₁X₂X₃ and 2-6 nucleoside-modified regions at the 3′ end, or there is part or all of nucleoside modifications.

In another preferred embodiment, the gap region Gap 1 comprises at least L_{g1} consecutive nucleotides without nucleoside modification, wherein L_{g1} is a positive integer of 5-14, preferably 8, 9, 10, 11, 12, 13 or 14; more preferably, 10, 11, 12 or 13.

In another preferred embodiment, the gap region Gap2 comprises at least L_{g2} consecutive nucleotides without nucleoside modification, wherein L_{g2} is a positive integer of 5-11, preferably 8-10, and more preferably 8, 9 or 10.

In another preferred embodiment, the oligonucleotide is complementary to the sequence shown in SEQ ID NO.25 or is completely complementary to a base sequence having at least 96% (preferably, at least 98%; more preferably, 100%) homology to the sequence shown in SEQ ID NO.25.

In another preferred embodiment, the oligonucleotide is any one oligonucleotide shown in SEQ ID NO.3-6, or the oligonucleotide in which one or more Ts are substituted with U in any one oligonucleotide shown in SEQ ID NO.3-6.

In another preferred embodiment, the oligonucleotide is the oligonucleotide shown in SEQ ID NO.6, or the oligonucleotide in which one or more Ts are substituted with U in the oligonucleotide shown in SEQ ID NO.6.

In another preferred embodiment, the oligonucleotide is shown in SEQ ID NO.2 (i.e., the T at the 3′ end of the oligonucleotide shown in SEQ ID NO.6 is substituted with U) or SEQ ID NO.6.

In another preferred embodiment, the modification of internucleoside bonds means that part or all (preferably, at least 90%; more preferably, all) of phosphodiester bonds(—OP(OH)(═O)O— or —OP(O⁻) (═O)O—) in the oligonucleotide are substituted with phosphorothioate bonds (—OP(OH) (═S)O— or —OP(O⁻) (═S)O—) or phosphorodithioate bonds (preferably, phosphorothioate bond).

In another preferred embodiment, the internucleoside bond of the modified oligonucleotide is phosphorothioate bond or phosphorodithioate bond.

In another preferred embodiment, the internucleoside bond of the modified oligonucleotide is phosphorothioate bond.

In another preferred embodiment, the nucleoside modification is the glycosyl modification and the optional base modification; wherein the sugar is ribose or deoxyribose.

In another preferred embodiment, the 2′-O-methylated glycosyl modification means that the group in position 2 of the glycosyl group is -O-methyl (i.e., 2′-O-methyl).

In another preferred embodiment, the 2′-O-methoxyethylated glycosyl modification means that the group in group 2 of the glycosyl group is -O-methoxyethyl (i.e., 2′-O-methoxyethyl).

In another preferred embodiment, the oligonucleotide is modified, and the oligonucleotide is divided into S1 segment, S2 segment, S3 segment, S4 segment and S5 segment in the order of 5′ to 3′, i.e., the modified oligonucleotide is shown as 5′-S1-S2-S3-S4-S5-3′;
wherein,
each nucleoside in the S1 segment, the S3 segment and the S5 segment is a modified nucleoside (preferably, the modified nucleoside comprises a modified sugar and a modified or unmodified base);
each nucleoside in the S2 segment and the S4 segment is an unmodified nucleoside;
various nucleoside moieties of the oligonucleotide are independently connected by phosphorodiester bonds or phosphorothioate bonds (preferably, all are connected by phosphorothioate bonds).

In another preferred embodiment, the internucleoside bond in the modified oligonucleotide is at least partially a modified internucleoside bond (i.e., phosphorothioate internucleoside bond).

In another preferred embodiment, all the internucleoside bonds in the modified oligonucleotide are modified internucleoside bonds (i.e., phosphorothioate internucleoside bond).

In another preferred embodiment, the S1 segment corresponds to a region of 2-6 nts at the 5′ end that has modifications.

In another preferred embodiment, the S2 segment corresponds to consecutive nucleotides including Lg1 non-nucleoside modifications in the gap region Gap1.

In another preferred embodiment, the S3 segment corresponds to the modifications present at 2-3 nts in X₁X₂X₃ and optionally modifications at 1, 2 or 3 extending from the 3′ end and/or 5′ end of X1X2X3 .

In another preferred embodiment, the S4 segment corresponds to consecutive nucleotides including Lg₂ non-nucleotide modifications in the gap region Gap2, wherein Lg₂ is a positive integer of 5-11, preferably 8-10, and more preferably 8, 9 or 10.

In another preferred embodiment, the S5 segment corresponds to a region with 2-6 nts at the 3′ end.

In another preferred embodiment, the S1 segment has a length of 3, 4, or 5 nts, the S2 segment has a length of 8, 9, 10, 11, 12, 13, or 14 nts, the S3 segment has a length of 1, 2, 3, or 4 nts, the S4 segment has a length of 8, 9 or 10 nts, and/or the S5 segment has a length of 3, 4 or 5 nts.

In another preferred embodiment, the oligonucleotide is any one oligonucleotide shown in SEQ ID NO.3-6 or an oligonucleotide in which each is optionally substituted with U in any one oligonucleotide shown in SEQ ID NO.3-6; wherein the S1 segment has a length of 3, 4, or 5 nts, the S2 segment has a length of 8, 9, 10, 11, 12, or 13 nts, the S3 segment has a length of 2, 3, or 4 nts, the S4 segment has a length of 8, 9 or 10 nts, and/or the S5 segment has a length of 4 or 5 nts.

In another preferred embodiment, the oligonucleotide is an oligonucleotide shown in SEQ ID NO.6 or an oligonucleotide in which each T in the oligonucleotide is optionally substituted with U as shown in SEQ ID NO.6, wherein the S1 segment has a length of 4 nts, the S2 segment has a length of 13 nts, the S3 segment has a length of 2 nts, the S4 segment has a length of 9 nts, and/or the S5 segment has a length of 4 nts.

In another preferred embodiment, in the segment S1, segment S3 and segment S5, the modified nucleosides are each independently glycosyl- modified nucleosides, or glycosyl and base-modified nucleosides.

In another preferred embodiment, the glycosyl- modified nucleosides are nucleosides whose glycosyl contains a 2′-O-methyl group.

In another preferred embodiment, the glycosyl and base-modified nucleosides are nucleosides whose glycosyl contains a 2′-O-methyl group and base is 5-methylcytosine.

In another preferred embodiment, the oligonucleotide is shown in SEQ ID NO.2 (i.e., the T at the 3′ end of the oligonucleotide shown in SEQ ID NO.6 is substituted with U); and
the S1 segment has a length of 4 nts, and each nucleoside therein is a modified nucleoside;
the S2 segment has a length of 13 nts, and each nucleoside therein is an unmodified nucleoside;
the S3 segment has a length of 2 nts, and each nucleoside therein is a modified nucleoside;
the S4 segment has a length of 9 nts, and each nucleoside therein is an unmodified nucleoside; and/or
the S5 segment has a length of 4 nts, and each nucleoside therein is a modified nucleoside.

In another preferred embodiment, in S1 segment, S3 segment and S5 segment, the modified nucleosides are each independently glycosyl-modified nucleosides, or moieties of glycosyl and base-modified nucleosides when the base is cytosine;
wherein, the glycosyl-modified nucleosides are nucleosides whose glycosyl contains a 2′-O-methyl group, and the glycosyl and base-modified nucleosides are nucleosides whose glycosyl contains a 2′-O-methyl group and base is 5-methylcytosine.

In another preferred embodiment, the compound is a modified oligonucleotide, and the compound is selected from the group consisting of:
mG*mA*mC*mG*T*G*C*A*G*A*G*G*T*G*A*A*G*mC*mG*A*A*G*T*G*C*A*C* A*mC*mG*mG*mU (PA0020); or mG*mA*(5Me-mC)*mG*T*G*C*A*G*A*G*G*T*G*A*A*G*(5Me-mC)*mG*A*A*G*T*G* C*A*C*A*(5Me-mC)*mG*mG*mU (PA0020C)
wherein, A, T, G and C represent unmodified nucleosides; mA, mU, mG and mC represent nucleosides modified by 2′-O-methylation; * represent phosphorothioate bond; and (5Me-mC) represent 2′-m ethoxy-5 -methylcytidine.

In a second aspect of the present invention, a pharmaceutical composition is provided, wherein the pharmaceutical composition comprises the compound as described in the first aspect, or a pharmaceutically acceptable salt, a hydrate or a solvate, and a pharmaceutically acceptable carrier thereof.

In a third aspect of the present invention, the use of the compound as described in the first aspect, or a pharmaceutically acceptable salt, a hydrate or a solvate thereof, or the pharmaceutical composition as described in the second aspect in the preparation of drugs for treating and/or preventing hepatitis B (HBV) or hepatitis D (HDV) virus-related diseases.

In another preferred embodiment, the diseases include one or more of the following diseases: diseases associated with hepatitis B virus infection, diseases associated with co-infection of hepatitis B virus and hepatitis D virus.

In another preferred embodiment, the disease may be an acute disease or a chronic disease.

In another preferred embodiment, the disease includes viral hepatitis B, viral hepatitis D, liver fibrosis, liver cirrhosis, hepatocellular carcinoma (HCC), or a combination thereof.

In another preferred embodiment, the disease includes an acute or a chronic liver disease.

In a fourth aspect of the present invention, a method for treating and/or preventing diseases related to hepatitis B virus or hepatitis D virus is provided. The method comprises the following steps: administering the compound as described in the first aspect, or a pharmaceutically acceptable salt, a hydrate or a solvate thereof, or the pharmaceutical composition as described in the second aspect to a subject in need at a safe and effective dose.

In another preferred embodiment, the disease is as defined in the third aspect.

In another preferred embodiment, the method is to administer the compound as described in the first aspect, or a pharmaceutically acceptable salt, a hydrate or a solvate thereof, or the pharmaceutical composition as described in the second aspect to a subject in need at a safe and effective dose by intravenous injection and/or subcutaneous injection.

In another preferred embodiment, the subject is a mammal; preferably, the subject is selected from human, rat, mouse, or a combination thereof.

In a fifth aspect of the present invention, a method for regulating the expression of HBV DNA/RNA, HBsAg and/or HBeAg is provided. The method comprises the following steps: exposing a subject to the compound as described in the first aspect, or a pharmaceutically acceptable salt, a hydrat or a solvate thereof, thereby regulating the expression of HBV DNA/RNA, HBsAg and/or HBeAg.

In another preferred embodiment, the method is non-therapeutic in vitro.

In another preferred embodiment, the subject is cells.

In another preferred embodiment, the regulation refers to inhibiting the expression of HBV DNA/RNA, HBsAg and/or HBeAg, or reducing the extracellular HBV RNA and/or HBeAg level (for example, in a cell culture medium).

It should be understood that within the scope of the present invention, the foregoing technical features and the technical features specifically described below (for example, in the examples) can be combined with each other to constitute new or preferred technical solutions. It is not describe again due to space limitation.

### BRIEF DESCRIPTION OF THE DRAWINGS

FIG. 1 shows the antiviral effect of liposome-transfected antisense oligonucleotides with different sequences in the HepG2.2.1.5 cell line, which is evaluated by detecting the concentration of HBsAg secreted in the cell supernatant at the end of the test.

FIG. 2 shows the antiviral effects of different concentrations of antisense oligonucleotides PA0020 and PA0021 transfected with liposomes in the HepG2.2.1.5 cell line, which is evaluated by detecting the concentration of HBsAg secreted in the cell supernatant at the end of the test.

FIG. 3 shows the antiviral effect of PA0020 administered by intraperitoneal injection once a week in increasing doses of 30 mg/kg, 60 mg/kg, and 90 mg/kg in AAV-HBV mouse models, which is evaluated by detecting the serum HBV-DNA by qPCR and detecting the serum HBsAg and HBeAg by ELISA at the end of the treatment.

FIG. 4 shows the antiviral effect of PA0020C at a dose of 90 mg/kg by intraperitoneal injection once a week in in AAV-HBV mouse models, which is evaluated by detecting the serum HBV-DNA by qPCR and detecting the serum HBsAg by ELISA at the end of the treatment.

FIG. 5 shows the relative inhibition rate of antisense oligonucleotides with different Gap modification patterns on the expression and secretion of HBsAg by HepG2.2.1.5.

### DETAILED DESCRIPTION

After long-term and in-depth studies, the inventors unexpectedly found that the antisense oligonucleotide with a length of only 24-40 nts that can be complementary to the conserved sequence of the pan-genotypehepatitis B virus (i.e., the oligonucleotide including the core sequence shown in SEQ ID NO.1) has remarkable antiviral activity. In particular, the Double-Gapantisense oligonucleotide whose sequence is SEQ ID NO.6 (or wherein T is substituted with U) (i.e., the oligonucleotide modified by the specific modification method described herein) can also significantly inhibit the replication of hepatitis B virus DNA at the animal level and significantly reduce the concentrations of HBsAg and HBeAg in serum. In the present invention, the antisense oligonucleotide can clearly target viral RNA, thereby reducing the expression of viral gene products at the transcriptional level; therefore, it is very suitable for combining with other antiviral therapies in the field, and has the prospect of functionally curing hepatitis B. Based on the above findings, the inventors have completed the present invention.

### Terms

The term “oligonucleotide” refers to an oligomer of nucleotides in ribonucleic acid (RNA) and/or deoxyribonucleic acid (DNA). The term includes oligonucleotides consisting of modified or unmodified nucleobases, modified or unmodified sugars (ribose or deoxyribose) and modified or unmodifiedinternucleoside bonds (phosphodiester bonds), and oligonucleotides whose one or more of bases can be optionally substituted (for example, substituting T with a modified or unmodified U), as well as functionally similar oligonucleotides with non-naturally occurring moieties. Particularly, modified or substituted oligonucleotides may be preferred over the natural form due to desirable properties such as, for example, reduced immunoreactivity, enhanced cellular uptake, increased affinity to nucleic acid targets, and/or increased stabilization against nuclease-mediated degradation. In the present invention, oligonucleotides can be single-stranded or double-stranded, including single-stranded molecules, such as antisense oligonucleotide (ASO), and aptamers and miRNA, etc., and double-stranded molecules, such as small interfering RNA (siRNA) or small hairpin RNA (shRNA). The oligonucleotide may comprise various modifications, for example, stabilizing modifications, and thus at least one modification may be carried out or at least one modifying group is included on the phosphodiester bonds (part or all) and/or on the sugar and/or the base. For example, an oligonucleotide may be subject to, including but not limited to, one or more modifications, or subject to complete modification so as to contain all bonds or sugars or bases with such modifications (i.e., every phosphodiester bond, sugar or base that makes up the oligonucleotide is unmodified, or partially or completely modified). In the present invention, the modified internucleoside bond may be phosphorothioate bond and/or phosphorodithioate bond. Other modifications useful for the present invention include, but are not limited to, modifications at the 2′ position of the sugar, including 2′-O-alkyl modifications (such as 2′O-methyl modification, 2′O-methoxyethyl (2 ‘MOE)), 2’-amino modification, 2′-halo modification (such as 2′-fluoro substitution); acyclic nucleotide analogs. Other modifications at the 2′ position are also well known in the art and can be used, such as locked nucleic acids. Specifically, oligonucleotides have modified bonds throughout or each modified bond, for example, phosphorothioate; having 3′-cap and/or 5′-cap: including 3′-5′ bonds at the end. Base modifications may include 5′methylation of cytosine bases (5′methylcytosine) and/or 4′thiolation of uracil bases (4′thiouracil). When the synthetic conditions are chemically compatible, different chemically compatible modified bonds can be combined, for example, oligonucleotides with phosphorothioate bond, 2′ ribose modification (e.g., 2′O-methylation) and modified bases (e.g., 5′ methylcytosine). Oligonucleotides can be further completely modified by making use of these different modifications (e.g., each phosphorothioated bond, each 2′ modified ribose, and each modified base).

For the sake of brevity, unless otherwise specified, an oligonucleotide expressed herein in the form of a DNA/RNA sequence or defined in a manner, for example, as shown in SEQ ID NO. 6, includes the modified or unmodified instances of the oligonucleotide having the sequence.

The term “antisense oligonucleotide” refers to a single-stranded oligonucleotide having a nucleic acid base sequence that allows hybridization to a corresponding segment of a target nucleic acid.

As used herein, the term “nt” refers to nucleotides.

The term “complementary” refers to the ability of the nucleic acid base sequence of the antisense oligonucleotide to perform accurate base pairing with the corresponding nucleic acid base sequence in the target nucleic acid, and is mediated by hydrogen bonding between the corresponding nucleic acid bases, for example, adenine pairs with thymine (or uracil), and guanine pairs with cytosine.

In the present invention, the term “comprises/comprise”, “includes/include” or “contain/contains” means that various components can be used together in the mixture or composition of the present invention. Accordingly, the terms “consisting essentially of” and “consisting of” are included in the term “comprising”.

In the present invention, the term “pharmaceutically acceptable” ingredient refers to a substance that is suitable for human and/or animal without undue adverse side effects (such as toxicity, irritation and allergic reaction), i.e., a substance that has a reasonable benefit/risk ratio.

In the present invention, the term “effective dose” refers to a dose of a therapeutic agent that treats, alleviates or prevents a target disease or condition, or a dose that exhibits a detectable therapeutic or preventive effect. The precise and effective dose for a subject depends on the size and health conditions of the subject, the nature and extent of the disease, and therapeutic agents and/or a combination of therapeutic agents chosen for administration. Therefore, it is useless to specify an exact and effective dose in advance. However, the effective dose can be determined by routine experimentation, within the judgment of the clinician, for a given situation.

As used herein, the term “pharmaceutically acceptable salt” refers to a salt formed by the compound of the present invention and a base, which is suitable for use as a medicine.

Unless otherwise specified, in the present invention, all appearing compounds are intended to include all possible optical isomers, such as single chiral compounds, or mixtures (i.e., racemates) of various chiral compounds. In all compounds of the present invention, each chiral carbon atom may optionally be in R configuration or S configuration, or a mixture of R and S configurations.

Some compounds of the present invention may be crystallized or recrystallized from water or various organic solvents, in such cases, various solvates may be formed. The solvates of the present invention include stoichiometric solvates, for example, hydrates, etc., as well as compounds containing variable amounts of water formed when prepared by low-pressure sublimation drying.

The present invention provides a method, a compound and a composition for regulating the expression of HBV DNA/RNA, HBsAg and HBeAg. In an embodiment, the compound suitable for regulating the expression of HBV DNA/RNA, HBsAg and HBeAg is antisense oligonucleotide.

In some embodiments, regulation can be performed in cells. In some embodiments, regulation is performed in an animal. In some embodiments, the animal is a human. In some embodiments, HBV RNA levels are reduced. In some embodiments HBV-DNA levels are reduced. In some embodiments HBsAg levels are reduced. In some embodiments, HBeAg levels are reduced. Therefore, the reduction occurs in a dose- and time-dependent manner.

Also provided are methods, compounds and compositions suitable for the prevention, treatment and amelioration of diseases, disorders and conditions. In some embodiments, the HBV-related diseases, disorders and conditions are acute or chronic liver diseases. In some embodiments, the liver diseases, disorders and conditions include viral hepatitis B, viral hepatitis D, liver fibrosis, liver cirrhosis, hepatocellular carcinoma (HCC), and etc.

In some embodiments, the treatment method comprises administering an HBV antisense oligonucleotide by intravenous or subcutaneous injection to a subject in need thereof.

### Oligonucleotide (Antisense Oligonucleotide)

In the present invention, the inventors unexpectedly discover oligonucleotides capable of inhibiting expression of virus proteins such as DNA/RNA of hepatitis B virus (HBV) surface antigen (HBsAg) and e antigen (HBeAg)(or called antisense oligonucleotides). Typically, the antisense oligonucleotide provided by the present invention can be complementary to a pan-genotype conserved sequence of the hepatitis B virus genome. For example, the antisense oligonucleotide provided by the present invention can be complementary to, for example, a segment pan-genotype conserved sequence shown in SEQ ID NO.25 in the hepatitis B virus genome (genotype D) shown in SEQ ID NO.24, or sequences with more than 96% or more than 98% homology with the sequence.

| | |
|---|---|
| SEQ ID NO.24 | TTCCACAACC TTCCACCAAA CTCTGCAAGA TCCCAGAGTG AGAGGCCTGT |
| | ATTTCCCTGC TGGTGGCTCC AGTTCAGGAA CAGTAAACCC TGTTCTGACT |
| | ACTGCCTCTC CCTTATCGTC AATCTTCTCG AGGATTGGGG ACCCTGCGCT |
| | GAACATGGAG AACATCACAT CAGGATTCCT AGGACCCCTT CTCGTGTTAC |
| | AGGCGGGGTT TTTCTTGTTG ACAAGAATCC TCACAATACC GCAGAGTCTA |
| | GACTCGTGGT GGACTTCTCT CAATTTTCTA GGGGGAACTA CCGTGTGTCT |
| | TGGCCAAAAT TCGCAGTCCC CAACCTCCAA TCACTCACCA ACCTCTTGTC |
| | CTCCAACTTG TCCTGGTTAT CGCTGGATGT GTCTGCGGCG TTTTATCATC |
| | TTCCTCTTCA TCCTGCTGCT ATGCCTCATC TTCTTGTTGG TTCTTCTGGA |
| | CTATCAAGGT ATGTTGCCCG TTTGTCCTCT AATTCCAGGA TCCTCAACAA |
| | CCAGCACGGG ACCATGCCGG ACCTGCATGA CTACTGCTCA AGGAACCTCT |
| | ATGTATCCCT CCTGTTGCTG TACCAAACCT TCGGACGGAA ATTGCACCTG |
| | TATTCCCATC CCATCATCCT GGGCTTTCGG AAAATTCCTA TGGGAGTGGG |
| | CCTCAGCCCG TTTCTCCTGG CTCAGTTTAC TAGTGCCATT TGTTCAGTGG |
| | TTCGTAGGGC TTTCCCCCAC TGTTTGGCTT TCAGTTATAT GGATGATGTG |
| | GTATTGGGGG CCAAGTCTGT ACAGCATCTT GAGTCCCTTT TTACCGCTGT |
| | TACCAATTTT CTTTTGTCTT TGGGTATACA TTTAAACCCT AACAAAACAA |
| | AGAGATGGGG TTACTCTCTA AATTTTATGG GTTATGTCAT TGGATGTTAT |
| | GGGTCCTTGC CACAAGAACA CATCATACAA AAAATCAAAG AATGTTTTAG |
| | AAAACTTCCT ATTAACAGGC CTATTGATTG GAAAGTATGT CAACGAATTG |
| | TGGGTCTTTT GGGTTTTGCT GCCCCTTTTA CACAATGTGG TTATCCTGCG |
| | TTGATGCCTT TGTATGCATG TATTCAATCT AAGCAGGCTT TCACTTTCTC |
| | GCCAACTTAC AAGGCCTTTC TGTGTAAACA ATACCTGAAC CTTTACCCCG |
| | TTGCCCGGCA ACGGCCAGGT CTGTGCCAAG TGTTTGCTGA CGCAACCCCC |
| | GGCTCCTCTG CCGATCCATA CTGCGGAACT CCTAGCCGCT TGTTTTGCTC |
| | ACTGGCTGGG GCTTGGTCAT GGGCCATCAG CGCATGCGTG GAACCTTTTC |
| | GCAGCAGGTC TGGAGCAAAC ATTATCGGGA CTGATAACTC TGTTGTCCTA |
| | TCCCGCAAAT ATACATCGTT TCCATGGCTG CTAGGCTGTG CTGCCAACTG |
| | GATCCTGCGC GGGACGTCCT TTGTTTACGT CCCGTCGGCG CTGAATCCTG |
| | CGGACGACCC TTCTCGGGGT CGCTTGGGAC TCTCTCGTCC CCTTCTCCGT |
| | CTGCCGTTCC GACCGACCAC GGGGCGCACC TCTCTTTACG CGGACTCCCC |
| | GTCTGTGCCT TCTCATCTGC CGGACCGTGT GCACTTCGCT TCACCTCTGC |
| | ACGTCGCATG GAGACCACCG TGAACGCCCA CCAAATATTG CCCAAGGTCT |
| | TACATAAGAG GACTCTTGGA CTCTCAGCAA TGTCAACGAC CGACCTTGAG |
| | GCATACTTCA AAGACTGTTT GTTTAAAGAC TGGGAGGAGT TGGGGGAGGA |
| | GATTAGGTTA AAGGTCTTTG TACTAGGAGG CTGTAGGCAT AAATTGGTCT |
| | GCGCACCAGC ACCATGCAAC TTTTTCACCT CTGCCTAATC ATCTCTTGTT |
| | CATGTCCTAC TGTTCAAGCC TCCAAGCTGT GCCTTGGGTG GCTTTGGGGC |
| | ATGGACATCG ACCCTTATAA AGAATTTGGA GCTACTGTGG AGTTACTCTC |
| | GTTTTTGCCT TCTGACTTCT TTCCTTCAGT ACGAGATCTT CTAGATACCG |
| | CCTCAGCTCT GTATCGGGAA GCCTTAGAGT CTCCTGAGCA TTGTTCACCT |
| | CACCATACTG CACTCAGGCA AGCAATTCTT TGCTGGGGGG AACTAATGAC |
| | TCTAGCTACC TGGGTGGGTG TTAATTTGGA AGATCCAGCG TCTAGAGACC |
| | TAGTAGTCAG TTATGTCAAC ACTAATATGG GCCTAAAGTT CAGGCAACTC |
| | TTGTGGTTTC ACATTTCTTG TCTCACTTTT GGAAGAGAAA CAGTTATAGA |
| | GTATTTGGTG TCTTTCGGAG TGTGGATTCG CACTCCTCCA GCTTATAGAC |
| | CACCAAATGC CCCTATCCTA TCAACACTTC CGGAGACTAC TGTTGTTAGA |
| | CGACGAGGCA GGTCCCCTAG AAGAAGAACT CCCTCGCCTC GCAGACGAAG |
| | GTCTCAATCG CCGCGTCGCA GAAGATCTCA ATCTCGGGAA TCTCAATGTT |
| | AGTATTCCTT GGACTCATAA GGTGGGGAAC TTTACTGGGC TTTATTCTTC |
| | TACTGTACCT GTCTTTAATC CTCATTGGAA AACACCATCT TTTCCTAATA |
| | TACATTTACA CCAAGACATT ATCAAAAAAT GTGAACAGTT TGTAGGCCCA |
| | CTCACAGTTA ATGAGAAAAG AAGATTGCAA TTGATTATGC CTGCCAGGTT |
| | TTATCCAAAG GTTACCAAAT ATTTACCATT GGATAAGGGT ATTAAACCTT |
| | ATTATCCAGA ACATCTAGTT AATCATTACT TCCAAACTAG ACACTATTTA |
| | CACACTCTAT GGAAGGCGGG TATATTATAT AAGAGAGAAA CAACACATAG |
| | CGCCTCATTT TGTGGGTCAC CATATTCTTG GGAACAAGAT CTACAGCATG |
| | GGGCAGAATC TTTCCACCAG CAATCCTCTG GGATTCTTTC CCGACCACCA |
| | GTTGGATCCA GCCTTCAGAG CAAACACCGC AAATCCAGAT TGGGACTTCA |
| | ATCCCAACAA GGACACCTGG CCAGACGCCA ACAAGGTAGG AGCTGGAGCA |
| | TTCGGGCTGG GTTTCACCCC ACCGCACGGA GGCCTTTTGG GGTGGAGCCC |
| | TCAGGCTCAG GGCATACTAC AAACTTTGCC AGCAAATCCG CCTCCTGCCT |
| | CCACCAATCG CCAGTCAGGA AGGCAGCCTA CCCCGCTGTC TCCACCTTTG |
| | AGAAACACTC ATCCTCAGGC CATGCAGTGG AA |
| SEQ ID NO.25 | CTGCCGGACCGTGTGCACTTCGCTTCACCTCTGCACGTCGCATGGA |

In addition, the present invention also provides modified antisense oligonucleotides. The preferred modified antisense oligonucleotide provided by the present invention is phosphorothioate-modified oligonucleotide, wherein the thio modification improves the stability of oligonucleotide in vivo, enhances plasma protein binding, and facilitates the distribution of oligonucleotide to target tissues such as the liver. The more preferred modified antisense oligonucleotide provided by the present invention is designed by Gapmer to modify the ribose of the 5′ and 3′-terminal nucleotides of the oligonucleotide, which improves the affinity with the target RNA, enhances the efficacy, and can reduce the effective dose and increase the safety window; meanwhile, the ribose modification at both ends further improves the stability of oligonucleotide. The most preferred antisense oligonucleotide provided by the present invention is double-Gap antisense oligonucleotide, which has carried out the modifications of the nucleotide ribose moiety at the 5′ and 3′ ends of the oligonucleotide and the bases of the cytosine nucleotide in the CpG sequence (i.e., cytosine (C)-phosphoric acid (p)-guanine (G)), thereby further increasing the affinity of oligonucleotide and reducing the off-target toxicity caused by the activation of innate immune Toll-like receptors.

In a specific embodiment, a compound comprising a modified or unmodified oligonucleotide is provided. The oligonucleotide provided by the present invention has a length of 24-40 nts (for example, 25-38 nts) (i.e., the oligonucleotide is composed of 25 to 38 linked nucleosides).

In another preferred embodiment, the oligonucleotide includes the core sequence shown in SEQ ID NO.1:

(SEQ ID NO. 1)
5'-GTGCAGAGGTGAAX1X2X3AAGTGCAC-3'.

In another preferred embodiment, the modification of the oligonucleotide can be nucleoside modification, for example, one or more of 2′-O-methylated glycosyl modification, 2′-O-methoxyethylated glycosyl modification, and methylation modification of the C-5 position of cytosine.

In another preferred embodiment, the modification of the oligonucleotide may be a modification of internucleoside bonds, for example, some or all of the internucleoside bonds in the oligonucleotide are substituted with phosphorothioate internucleoside bonds and/or phosphorodithioate internucleoside bonds.

In another preferred embodiment, the oligonucleotide is complementary to at least a part of the fragment in SEQ ID NO.25.

In another preferred embodiment, the oligonucleotide is complementary to at least a part of SEQ ID NO.25 and at least a part of SEQ ID NO.24.

In another preferred embodiment, the oligonucleotide is at least 96% complementary to SEQ ID NO.25.

In another preferred embodiment, the oligonucleotide is composed of a single-chain modified oligonucleotide.

In another preferred embodiment, the internucleoside bond of the oligonucleotide is a phosphorothioate internucleoside bond.

In another preferred embodiment, at least 3 non-adjacent nucleosides comprise modified sugars in the oligonucleotide.

In another preferred embodiment, the modified sugar comprises a 2′-O-methyl group.

In another preferred embodiment, the modified sugar comprises a 2′-O-methoxyethyl group.

In another preferred embodiment, the oligonucleotide comprises modified nucleic acid bases (preferably, the modified nucleic acid bases include 5-methylcytosine).

In another preferred embodiment, the oligonucleotide comprises five segments S1-S2-S3-S4-S5 in the order of 5′ to 3′ successively, wherein the S1 segment, the S3 segment and the S5 segment are composed of linked nucleosides, and wherein each nucleoside comprises a modified sugar and optionally a modified base; wherein the S2 segment and the S4 segment are composed of linked deoxynucleosides.

In another preferred embodiment, the oligonucleotide sequence is SEQ ID NO.3-6, wherein the S1 segment is composed of 3, 4 or 5 linked nucleosides comprising modified glycosyl groups and optionally modified bases, wherein the S2 segment is composed of 8, 9, 10, 11, 12 or 13 linked deoxynucleosides, wherein the S3 segment is composed of 2, 3 or 4 linked nucleosides comprising modified glycosyl groups and optionally modified bases, wherein the S4 segment is composed of 8, 9 or 10 linked deoxynucleosides, where the S5 segment is compose of 4 or 5 linked nucleosides comprising modified glycosyl groups and optionally modified bases.

In another preferred embodiment, the sequence of the oligonucleotide is SEQ ID NO.2 or SEQ ID NO.6, wherein the S1 segment is composed of 4 linked nucleosides comprising modified glycosyl groups, wherein the S2 segment is composed of 13 linked deoxynucleosides, wherein the S3 segment is composed of 2 linked nucleosides comprising modified sugars, wherein the S4 segment is composed of 9 linked deoxynucleosides, wherein the S5 segment is composed of 4 linked sugar-modified nucleosides.

In another preferred embodiment, the sugar modification contained in the S1 segment, the S3 segment and the S5 segment refers to 2′-O-methylation modification.

In another preferred embodiment, the sequence of the oligonucleotide is SEQ ID NO.6, wherein the S1 segment is composed of 4 linked nucleosides comprising modified sugars and modified bases, wherein the S2 segment is composed of 13 linked unmodified deoxynucleoside, wherein the S3 segment is composed of 2 linked nucleosides comprising modified glycosyl groups and optionally modified bases, wherein the S4 segment is composed of 9 linked unmodified deoxynucleosides, wherein the S5 segment is composed of 4 linked nucleosides comprising modified sugars and modified bases.

In another preferred embodiment, the sugar modification contained in the S1 segment, the S3 segment, and the S5 segment refers to 2′-O-methylation modification, and the modified base refers to 5-methylcytosine.

In another specific embodiment, the present invention provides an oligonucleotide, or an optical isomer, a pharmaceutically acceptable salt, a hydrate, or a solvate thereof, or the sequence of the antisense oligonucleotide is selected from SEQ ID NO.3-6, and one or more of Ts can be optionally substituted with U.

| | |
|---|---|
| SEQ ID NO.3 | ACGTGCAGAGGTGAAGCGA |
| | AGTGCACACGGTCCGGCAG |
| SEQ ID NO.4 | TCCATGCGACGTGCAGAGG |
| | TGAAGCGAAGTGCACACGG |
| SEQ ID NO.5 | ACGTGCAGAGGTGAAGCGA |
| | AGTGCACACGG |
| SEQ ID NO.6 | GACGTGCAGAGGTGAAGCG |
| | AAGTGCACACGGT |

In another preferred embodiment, the modified oligonucleotide is PA0020, the sequence thereof is SEQ ID No.2, i.e., the T at the 3′ end in SEQ ID NO.6 is substituted with mU that can also be used for base pairing, the sugar modification and base modification are: mG*mA*mC*mG*T*G*C*A*G*A*G*G*T*G*A*A*G*mC*mG*A*A*G*T*G*C*A*C*A*mC *mG*mG*mU, wherein A/T/G/C represents conventional unmodified DNA; mA/mU/mG/mC represents 2′methoxy modification, * represents phosphorothioate backbone.

In another preferred embodiment, the modified oligonucleotide is PA0020C, the sequence thereof is SEQ ID No.2, i.e., i.e., the T at the 3′ end in SEQ ID NO.6 is substituted with mU that can also be used for base pairing, the sugar modification and base modification are: mG*mA*(5Me-mC)*mG*T*G*C*A*G*A*G*G*T*G*A*A*G*(5Me-mC)*mG*A*A*G*T*G* C*A*C*A*(5Me-mC)*mG*mG*mU, wherein A/T/G/C represents conventional unmodified DNA; mA/mU/mG/mC represents 2′methoxy modification, * represents phosphorothioate backbone, (5Me-mC) represents 2′-methoxy-5-methylcytosine.

### Preparation of Antisense Oligonucleotide

The antisense oligonucleotide in the present invention can be prepared and synthesized by a conventional synthesis method in the oligonucleotide industry. For example, phosphorothioate bond can be synthesized by standard phosphoramidite chemical synthesis method on the equipment GE OP100, and 1,2-benzodithiol-3-one-1,1-dioxide is used to substitute iodine as an oxidizing reagent.

### Pharmaceutical Composition and Method of Administration

Since the compound of the present invention (or modified or unmodified oligonucleotide of the present invention) has excellent ability to inhibit the replication of hepatitis B virus DNA, the compound of the present invention and isomers (for example, optical isomers), a crystal form, a solvate, a pharmaceutically acceptable organic salt, and a pharmaceutical composition containing the compound of the present invention as the main active ingredient can be used for the treatment, prevention and alleviation of diseases related to or caused by hepatitis B virus (i.e., HBV) or diseases related to or caused by co-infection of hepatitis B virus and hepatitis D virus. These diseases can be acute or chronic. According to the prior art, the compound of the present invention can be used to treat the following diseases: hepatitis B; pancreatic cancer, liver cirrhosis and hepatocellular carcinoma, etc. (for example, pancreatic cancer, liver cirrhosis and hepatocellular carcinoma caused by chronic hepatitis B).

The pharmaceutical composition provided by the present invention comprises the compound of the present invention or other pharmaceutically acceptable forms (such as an optical isomer, a pharmaceutically acceptable salt, a hydrate or a solvate thereof), a pharmaceutically acceptable adjuvant, a diluent or a carrier thereof within the range of safe and effective dose. Wherein, a “safe and effective dose” means that the dose of the compound is sufficient to obviously improve the condition without causing serious side effects. Usually, the pharmaceutical composition contains 1-2000 mg of the compound of the present invention per dose, more preferably, 10-500 mg of the compound of the present invention per dose. Preferably, the “one dose” is an ampoule or a vial.

A “pharmaceutically acceptable carrier” refers to one or more compatible solid or liquid fillers or gel substances, which are suitable for human use and must have sufficient purity and sufficiently low toxicity. The “compatibility” herein means that the components of the composition can be blended with the compound of the present invention or they can be blended with each other without significantly reducing the efficacy of the compound. Some examples of the pharmaceutically acceptable carrier include cellulose and derivatives thereof (such as sodium carboxymethylcellulose, sodium ethylcellulose, cellulose acetate, etc.), gelatin, talc, solid lubricants (such as stearic acid , magnesium stearate), calcium sulfate, vegetable oils (such as soybean oil, sesame oil, peanut oil, olive oil, etc.), polyols (such as propylene glycol, glycerin, mannitol, sorbitol, etc.), emulsifiers (such as Tween®), wetting agents (such as sodium dodecyl sulfate), coloring agents, flavoring agents, stabilizers, antioxidants, preservatives, pyrogen-free water, etc.

### Method of Administration

The compound and the composition comprising the same of the present invention can be administered by any suitable means, for example, oral ingestion; oral inhalation; by subcutaneous, intravenous injection or infusion. The compound and the composition comprising the same of the present invention may be administered in the form of dosage unit formulations containing nontoxic pharmaceutically acceptable carriers or diluents or in the form of immediate- or sustained-release formulations.

Suggested effective dosing regimens for administering the antisense oligonucleotides of the present invention to humans follow the dosing regimens commonly used for other antisense oligonucleotides; in the prior art, the conventional parenteral administration of 100-500 mg of the compound per week is well established.

According to the disclosure presented herein, it is useful to treat subjects with HBV infection or HBV/HDV co-infection with pharmaceutically acceptable antisense oligonucleotide formulations.

### Main Advantages of the Present Invention:

(a) The compound or oligonucleotide provided by the present invention can effectively inhibit viral gene products at the transcriptional level in vitro, thereby significantly inhibiting hepatitis B virus antigens (such as HBsAg and HBeAg).
(b) The compounds or oligonucleotide provided by the present invention shows excellent ability to inhibit hepatitis B virus antigens (such as HBsAg and HBeAg) in vivo.
(c) The modified oligonucleotide provided by the present invention has the equivalent ability to inhibit hepatitis B virus antigens (such as HBsAg and HBeAg) as the unmodified oligonucleotide while improving the in vivo stability through modification.
(d) The compound provided by the present invention can significantly reduce HBsAg and surface antigens in serum in vivo.

The present invention will be further described below in conjunction with specific embodiments. It should be understood that these embodiments are only used to illustrate the present invention and are not intended to limit the scope of the present invention. The experimental method that does not indicate specific conditions in the following examples usually adopts conventional conditions, for example, the conditions described in Molecular Cloning: A Laboratory Manual by Sambrook et al. (New York: Cold Spring Harbor Laboratory Press, 1989), or conditions recommended by the manufacturer. Unless otherwise indicated, percentages and parts are percentages by weight and parts by weight.

It should be understood that those skilled in the art can obtain the oligonucleotides used in the examples according to the sequences and corresponding modifications described in the examples, conventional techniques (for example, standard solid-phase synthesis methods)in the prior art and using commercially available or synthesized raw materials (for example, modified or unmodified nucleosides) based on the methods in the prior art.

### EXAMPLE 1

Antiviral Effects of Antisense Oligonucleotides with Different Sequences in HepG2.2.1.5 Cell Line

The HepG2.2.1.5 cell line can stably express and replicate HBV virus, and secrete HBV virus particles, HBsAg and HBeAg into the cell supernatant. HepG2.2.1.5 cells were cultured in DMEM medium (Hyclone) containing 10% FBS (Gibico) and 400 ug/ml G418, and spread to a 96-well plate with 2×10⁴ cells/well after passage to the third generation. After 6 hours, oligonucleotide was transfected with riboFECT™CP transfection reagent (Guangzhou Ribobio) to a final concentration of 100 nM, after 48 hours, the medium was drawn and discarded, and PBS was added, allowed to stand for 5 min, then a fresh medium was used; 6 hours later after the medium change, the cell supernatant medium was taken and detected for HBsAg using an ELISA kit (DAAN Gene). The antisense oligonucleotides used are shown in Table 1.

TABLE 1
Description of oligonucleotides used in Example I

| | | | | Molecular | |
|---|---|---|---|---|---|
| | SEQ | | Length | weight | Oligonucleotide |
| Oligonucleotide | ID | Sequence | (nt) | (Da) | modification |
| PA0005 | SEQ ID | 5'-ACACACA | 40 | 12612 | Phosphorothioate |
| | NO. 16 | CACACACACA | | | internucleoside |
| | | CACACACACA | | | bond as the |
| | | CACACACACA | | | internucleoside |
| | | CAC-3' | | | bond |
| PA103 | SEQ ID | 5'-TCCATGC | 38 | 12394.7 | Phosphorothioate |
| | NO. 4 | GACGTGCAGA | | | internucleoside |
| | | GGTGAAGCGA | | | bond as the |
| | | AGTGCACACG | | | internucleoside |
| | | G-3' | | | bond |
| PA10336 | SEQ ID | 5'-CATGCGA | 36 | 11769.3 | Phosphorothioate |
| | NO. 12 | CGTGCAGAGG | | | internucleoside |
| | | TGAAGCGAAG | | | bond as the |
| | | TGCACACGG- | | | internucleoside |
| | | 3' | | | bond |
| PA10334 | SEQ ID | 5'-TGCGACG | 34 | 11134.9 | Phosphorothioate |
| | NO. 13 | TGCAGAGGTG | | | internucleoside |
| | | AAGCGAAGTG | | | bond as the |
| | | CACACGG-3' | | | internucleoside |
| | | | | | bond |
| PA10332 | SEQ ID | 5'-CGACGTG | 32 | 10469.5 | Phosphorothioate |
| | NO. 14 | CAGAGGTGAA | | | internucleoside |
| | | GCGAAGTGCA | | | bond as the |
| | | CACGG-3' | | | internucleoside |
| | | | | | bond |
| PA10330 | SEQ ID | 5'-ACGTGCA | 30 | 9819.1 | Phosphorothioate |
| | NO. 10 | GAGGTGAAGC | | | internucleoside |
| | | GAAGTGCACA | | | bond as the |
| | | CGG-3' | | | internucleoside |
| | | | | | bond |
| PA10325 | SEQ ID | 5'-CAGAGGT | 25 | 8174.1 | Phosphorothioate |
| | NO. 15 | GAAGCGAAGT | | | internucleoside |
| | | GCACACGG- | | | bond as the |
| | | 3' | | | internucleoside |
| | | | | | bond |
| PA102 | SEQ ID | 5'-ACGTGCA | 38 | 12419.7 | Phosphorothioate |
| | NO. 3 | GAGGTGAAGC | | | internucleoside |
| | | GAAGTGCACA | | | bond as the |
| | | CGGTCCGGCA | | | internucleoside |
| | | G-3' | | | bond |
| PA10236 | SEQ ID | 5'-ACGTGCA | 36 | 11745.3 | Phosphorothioate |
| | NO. 7 | GAGGTGAAGC | | | internucleoside |
| | | GAAGTGCACA | | | bond as the |
| | | CGGTCCGGC- | | | internucleoside |
| | | 3' | | | bond |
| PA10234 | SEQ ID | 5'-ACGTGCA | 34 | 11094.9 | Phosphorothioate |
| | NO. 8 | GAGGTGAAGC | | | internucleoside |
| | | GAAGTGCACA | | | bond as the |
| | | CGGTCCG-3' | | | internucleoside |
| | | | | | bond |
| PA10232 | SEQ ID | 5'-ACGTGCA | 32 | 10444.5 | Phosphorothioate |
| | NO. 9 | GAGGTGAAGC | | | internucleoside |
| | | GAAGTGCACA | | | bond as the |
| | | CGGTC-3' | | | internucleoside |
| | | | | | bond |
| PA10225 | SEQ ID | 5'-ACGTGCA | 25 | 8189.1 | Phosphorothioate |
| | NO. 11 | GAGGTGAAGC | | | internucleoside |
| | | GAAGTGCA- | | | bond as the |
| | | 3' | | | internucleoside |
| | | | | | bond |

The test results are shown in FIG. 1. As shown in the figure, PA103, PA10332, PA10330, PA10325, PA102, PA10236, PA10234, PA10232, and PA10225 significantly reduced the HBsgAg level in the cell supernatant medium, with an inhibition rate of 30% to 50%.

### EXAMPLE 2

### Antiviral Effect of Double-Gap Antisense Oligonucleotide in HepG2.2.15 Cell Line

The HepG2.2.1.5 cell line can stably express and replicate HBV virus, and secrete HBV virus particles, HBsAg and HBeAg into the cell supernatant. HepG2.2.1.5 cells were cultured in DMEM medium (Hyclone) containing 10% FBS (Gibico) and 400 ug/ml G418, and spread to a 96-well plate with 2×10⁴ cells/well after passage to the third generation. After 6 hours, oligonucleotides PA0020 and PA0021 were transfected with riboFECT™ CP transfection reagent (Guangzhou Ribobio) to final concentrations of 10 nM and 30 nM respectively, after 48 hours, the medium was drawn and discarded, and PBS was added, allowed to stand for 5 min, then a fresh medium was used; 6 hours later after the medium change, the cell supernatant medium was taken and detected for HBsAg using an ELISA kit (DAAN Gene). The antisense oligonucleotides used are shown in Table 2.

TABLE 2
Description of oligonucleotides
used in Example II

| Oligo- | | Molecular | |
|---|---|---|---|
| nucleo- | | weight | Oligonucleotide |
| tide | SEQ ID | (Da) | modification |
| PA0020 | SEQ ID | 10770.5 | mG*mA*mC*mG*T*G*C*A* |
| | NO. 2 | | G*A*G*G*T*G*A*A*G* |
| | | | mC*mG*A*A*G*T*G*C* |
| | | | A*C*A*mC*mG*mG*mU |
| PA0021 | SEQ ID | 10484.5 | G*A*C*G*T*G*C*A*G*A* |
| | NO. 6 | | G*G*T*G*A*A*G*C*G*A* |
| | | | A*G*T*G*C*A*C*A*C*G* |
| | | | G*T |

Note: A/T/G/C represented conventional unmodified deoxyribonucleosides; mA/mU/mG/mC represented glycosyl groups in nucleosides including 2′-O-methyl modification groups, * represented phosphorothioate backbone modification.

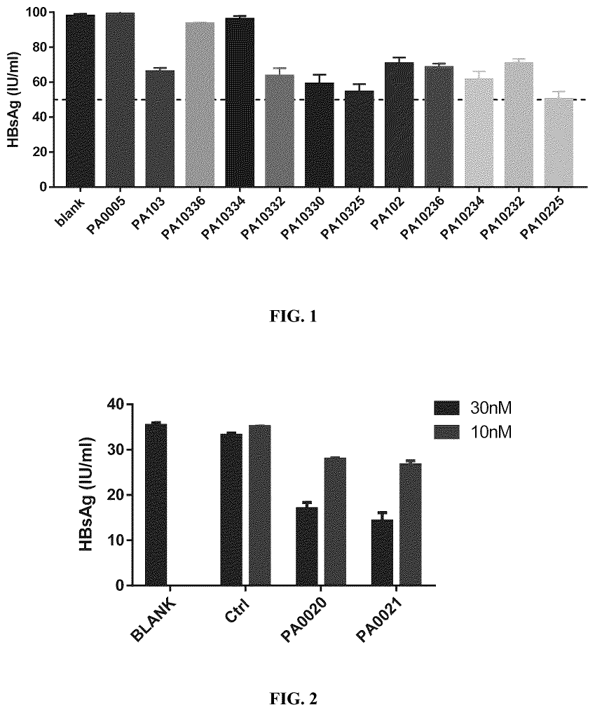

The test results are shown in FIG. 2. The results showed that, there was no significant difference in HBsAg concentration between the cell supernatant when no antisense oligonucleotide (BLANK) was transfected and that when only the transfection (Ctrl) reagent was added. The transfection with PA0020 and PA0021significantly reduced HBsgAg in cell supernatant medium in a concentration-dependent manner, and the inhibition rate could reach about 50% at a concentration of 30 nM.

### EXAMPLE 3

### Antiviral Effect of Double-Gap Antisense Oligonucleotide PA0020 in AAV-HBV Mouse Models

Antisense oligonucleotide PA0020 was dose-escalated in c57 mice infected with adeno-associated virus (AAV-HBV, FivePlus) carrying HBV1.3 ploidy and continuously replicating HBV-DNA and expressing HBV antigen, to evaluate the dose-dependent relationship of its antiviral activity. The oligonucleotide PA0020 used is shown in Table 3.

TABLE 3
Description of oligonucleotides
used in Example 3

| Oligo- | | Molecular | Purity | |
|---|---|---|---|---|
| nucleo- | | weight | HP | Oligonucleotide |
| tide | SEQ ID | (Da) | LC | modification |
| PA0020 | SEQ ID | 10774.5 | 91% | mG*mA*mC*mG*T* |
| | NO. 2 | | | G*C*A*G*A*G*G* |
| | | | | T*G*A*A*G*mC* |
| | | | | mG*A*A*G*T*G* |
| | | | | C*A*C*A*mC* |
| | | | | mG*mG*mU |

Note: A/T/G/C represented conventional unmodified deoxyribonucleosides; mA/mU/mG/mC represented glycosyl groups in nucleosides including 2′-O-methyl modification groups, * represented phosphorothioate backbone modification.

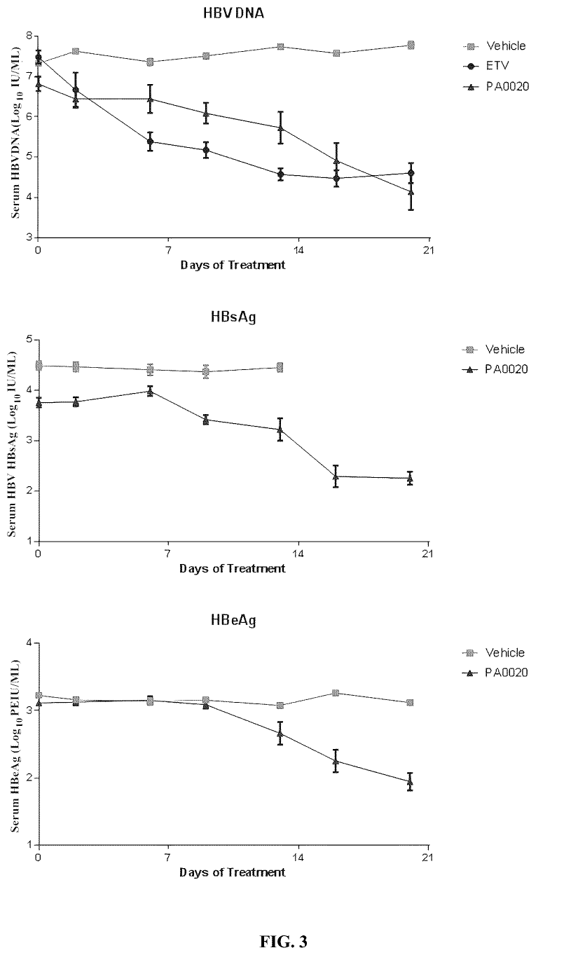

Male C57BL/6 mice were injected with 5×10¹⁰ rAAV8-1.3HBV (FivePlus) through the tail vein, to construct a mouse model of persistent hepatitis B infection. After confirming the stable replication of HBV virus, the mice were randomly divided into 3 groups according to the body weight (5 in each group). Animals in the group 1 were given normal saline by intragastric administration every day, those in group 2 were given entecavir (ETV) at a dose of 1 mg/kg/day by intragastric administration., and those in group 3 were given PA0020 by intraperitoneal injection once a week. The injection doses were 30 mg/kg, 60 mg/kg, and 90 mg/kg on day 0, day 7, and day 14, respectively. Blood was drawn twice a week, and the hepatitis B virus nucleic acid (HBV-DNA) load in the serum was analyzed by qPCR, and the concentrations of HBsAg and HBeAg in the serum were analyzed by ELISA. The curves were drawn, as shown in FIG. 3.

As shown in FIG. 3, HBsAg and HBV-DNA levels in the serum of animals in the control group showed stable fluctuations; HBV-DNA level in the serum of animals in the entecavir group continued to decrease, and the decrease was >2 log 10 on day 20 compared with that on day 0, i.e. a decrease >99%; the serum HBV-DNA level of animals in the PA0020 group continued to decrease, and the rate of decrease increased with the increase of the dose, and the decrease was >2 log₁₀ on day 20 compared with that on day 0, i.e. a decrease >99%. The HBsAg and HBeAg levels in the serum of animals in the PA0020 group continued to decrease from day 6, and the decrease was >1 log₁₀ on day 20 compared with that on day 0, i.e. a decrease >90%.

### EXAMPLE 4

### Antiviral Effect of Double-Gap Antisense Oligonucleotide PA0020C in AAV-HBV Mouse Models

Antisense oligonucleotide PA0020C was dose-escalated in c57 mice infected with adeno-associated virus (AAV-HBV, FivePlus) carrying HBV1.3 ploidy and continuously replicating HBV-DNA and expressing HBV antigen, to evaluate the dose-dependent relationship of its antiviral activity. The oligonucleotide PA0020C used is shown in Table 4.

TABLE 4
Description of the oligonucleotides
used in Example 4

| Oligo- | | Molecular | | |
|---|---|---|---|---|
| nucleo- | | weight | | Oligonucleotide |
| tide | SEQ ID | Da | Purity | modification |
| PA0020C | SEQ ID | 10815 | 94% | mG*mA*(5Me-mC)* |
| | NO. 2 | | | mG*T*G*C*A*G*A* |
| | | | | G*G*T*G*A*A*G* |
| | | | | (5Me-mC)*mG*A |
| | | | | *A*G*T*G*C*A* |
| | | | | C*A*(5Me-mC)* |
| | | | | mG*mG*mU |

Note: A/T/G/C represented conventional unmodified deoxyribonucleosides; mA/mU/mG/mC represented 2′-O-methyl modification, * represented phosphorothioate backbone modification, (5Me-mC) represented 2′-methoxy-5-methylcytosine.

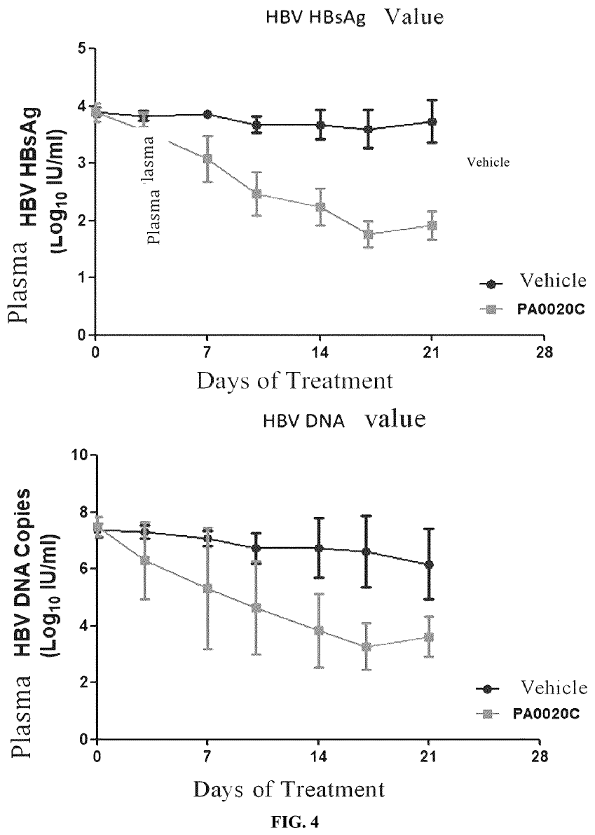

Male C57BL/6 mice were injected with 5×10¹⁰ rAAV8-1.3HBV (FivePlus) through the tail vein, to construct a mouse model of persistent hepatitis B infection. After confirming the stable replication of HBV virus, the mice were randomly divided into 2 groups according to the body weight (5 in the control group and 6 in the administration group). Animals in the control group (Vehicle) were given normal saline by intraperitoneal injection every day, and animals in the administration group were given PA0020C by intraperitoneal injection once a week. The injection dose was 90 mg/kg on day 0, day 7 and day 14. Blood was drawn twice a week, and the hepatitis B virus nucleic acid (HBV-DNA) load in the serum was analyzed by qPCR, and the concentration of HBsAg in the serum was analyzed by ELISA. The curves were drawn, as shown in FIG. 4.

As shown in FIG. 4, HBsAg and HBV-DNA levels in the serum of animals in the control group showed stable fluctuations; the HBV-DNA level in the serum of animals in the PA0020C group continued to decrease, and the decrease was >3 log 10 compared with that on day 0 at the end of administration, i.e., a decrease >99.9%. The serum HBsAg level of animas in the PA0020C group continued to decrease from day 0, and the decrease rate was >2 log 10 compared with that on day 0 at the end of administration, i.e. a decrease>90%. Therefore, PA0020C can not only reduce the HBV-DNA level, but also reduce the surface antigen. In the future, it is expected to achieve the purpose of functionally curing hepatitis B in the clinic.

### EXAMPLE 5

### Antiviral Effect of Double-Gap Antisense Oligonucleotide in HepG2.2.15 Cell Line

The HepG2.2.1.5 cell line can stably express and replicate HBV virus, and secrete HBV virus particles, HBsAg and HBeAg into the cell supernatant. HepG2.2.1.5 cells were cultured in DMEM medium (Hyclone) containing 10% FBS (Gibico) and 400 ug/ml G418, and spread to a 96-well plate with 2×10⁴ cells/well after passage to the third generation. After 6 hours, oligonucleotides were transfected with riboFECT™CP transfection reagent (Guangzhou Ribobio) to final concentrations of 30 nM, after 48 hours, the medium was drawn and discarded, and PBS was added, allowed to stand for 5 min, then a fresh medium was used; 6 hours later after the medium change, the cell supernatant medium was taken and detected for HBsAg using an ELISA kit (DAAN Gene). The antisense oligonucleotides used are shown in Table 5.

TABLE 5
Description of oligonucleotides used in Example 5

| | | | Gap modification |
|---|---|---|---|
| | | | mode |
| Oligonucleotide | Sequence | SEQ ID | S1-S2-S3-S4-S5 |
| PA0020 | mG*mA*mC*mG*T*G*C*A*G*A*G*G*T*G* | SEQ ID NO. 2 | 4-13-2-9-4 |
| | A*A*G*mC*mG*A*A*G*T*G*C*A*C*A*mC | | |
| | kmG*mG*mU | | |
| PA0041 | mG*mA*mC*mG*T*G*C*A*G*A*G*G*T*G* | SEQ ID NO. 2 | 4-24-4 |
| | A*A*G*C*G*A*A*G*T*G*C*A*C*A*mC*m | | |
| | G*mG*mU | | |
| PA0042 | mG*mA*mC*mG*mU*G*C*A*G*A*G*G*T* | SEQ ID NO. 17 | 5-22-5 |
| | G*A*A*G*C*G*A*A*G*T*G*C*A*C*mA*m | | |
| | C*mG*mG*mU | | |
| PA0043 | mG*mA*mC*mG*T*G*C*A*G*A*G*G*T*G* | SEQ ID NO. 2 | 4-12-2-10-4 |
| | A*A*mG*mC*G*A*A*G*T*G*C*A*C*A*mC | | |
| | *mG*mG*mU | | |
| PA0044 | mG*mA*mC*mG*T*G*C*A*G*A*G*G*T*G* | SEQ ID NO. 2 | 4-11-3-10-4 |
| | A*mA*mG*mC*G*A*A*G*T*G*C*A*C*A*m | | |
| | C*mG*mG*mU | | |
| PA0045 | mG*mA*mC*mG*T*G*C*A*G*A*G*G*T*G* | SEQ ID NO. 2 | 4-10-4-10-4 |
| | mA*mA*mG*mC*G*A*A*G*T*G*C*A*C*A* | | |
| | mC*mG*mG*mU | | |
| PA0046 | mG*mA*mC*mG*T*G*C*A*G*A*G*G*T*m | SEQ ID NO. 2 | 4-9-5-10-4 |
| | G*mA*mA*mG*mC*G*A*A*G*T*G*C*A*C* | | |
| | A*mC*mG*mG*mU | | |
| PA0047 | mG*mA*mC*mG*T*G*C*A*G*A*G*G*T*G* | SEQ ID NO. 2 | 4-10-5-9-4 |
| | mA*mA*mG*mC*mG*A*A*G*T*G*C*A*C* | | |
| | A*mC*mG*mG*mU | | |
| PA0048 | mG*mA*mC*mG*T*G*C*A*G*A*G*G*T*m | SEQ ID NO. 2 | 4-9-6-9-4 |
| | G*mA*mA*mG*mC*mG*A*A*G*T*G*C*A* | | |
| | C*A*mC*mG*mG*mU | | |
| PA0049 | mG*mA*mC*mG*mU*G*C*A*G*A*G*G*T* | SEQ ID NO. 17 | 5-9-4-9-5 |
| | G*mA*mA*mG*mC*G*A*A*G*T*G*C*A*C* | | |
| | mA*mC*mG*mG*mU | | |
| PA0050 | mG*mA*mC*mG*mU*G*C*A*G*A*G*G*T* | SEQ ID NO. 17 | 5-8-5-9-5 |
| | mG*mA*mA*mG*mC*G*A*A*G*T*G*C*A* | | |
| | C*mA*mC*mG*mG*mU | | |
| PA0051 | mG*mA*mC*mG*mU*G*C*A*G*A*G*G*T* | SEQ ID NO. 17 | 5-9-5-8-5 |
| | G*mA*mA*mG*mC*mG*A*A*G*T*G*C*A* | | |
| | C*mA*mC*mG*mG*mU | | |
| PA0052 | mG*mA*mC*mG*mU*G*C*A*G*A*G*G*T* | SEQ ID NO. 17 | 5-9-4-10-4 |
| | G*mA*mA*mG*mC*G*A*A*G*T*G*C*A*C* | | |
| | A*mC*mG*mG*mU | | |
| PA0053 | mG*mA*mC*mG*T*G*C*A*G*A*G*G*T*m | SEQ ID NO. 2 | 4-9-4-10-5 |
| | G*mA*mA*mG*C*G*A*A*G*T*G*C*A*C* | | |
| | mA*mC*mG*mG*mU | | |
| PA0054 | mG*mA*mC*mG*T*G*C*A*G*A*G*G*T*G* | SEQ ID NO. 2 | 4-10-4-9-5 |
| | mA*mA*mG*mC*G*A*A*G*T*G*C*A*C*m | | |
| | A*mC*mG*mG*mU | | |
| PA0055 | mG*mA*mC*mG*mU*G*C*A*G*A*G*G*T* | SEQ ID NO. 17 | 5-10-4-9-4 |
| | G*A*mA*mG*mC*mG*A*A*G*T*G*C*A*C* | | |
| | A*mC*mG*mG*mU | | |
| PA0056 | mG*mA*mC*mG*mU*G*C*A*G*A*G*G*T* | SEQ ID NO. 17 | 5-9-3-10-5 |
| | G*mA*mA*mG*C*G*A*A*G*T*G*C*A*C* | | |
| | mA*mC*mG*mG*mU | | |
| PA0057 | mG*mA*mC*mG*mU*G*C*A*G*A*G*G*T* | SEQ ID NO. 17 | 5-10-3-9-5 |
| | G*A*mA*mG*mC*G*A*A*G*T*G*C*A*C*m | | |
| | A*mC*mG*mG*mU | | |
| PA0058 | mG*mA*mC*mG*mU*G*C*A*G*A*G*G*T* | SEQ ID NO. 17 | 5-10-2-10-5 |
| | G*A*mA*mG*C*G*A*A*G*T*G*C*A*C*mA | | |
| | *mC*mG*mG*mU | | |
| PA0059 | mG*mA*mC*mG*mU*G*C*A*G*A*G*G*T* | SEQ ID NO. 18 | 5-10-5 |
| | G*A*mA*mG*mC*mG*mA | | |
| PA0060 | mU*mG*mA*mA*mG*C*G*A*A*G*T*G*C* | SEQ ID NO. 19 | 5-10-5 |
| | A*C*mA*mC*mG*mG*mU | | |
| PA0061 | mG*mU*mG*mA*mA*G*C*G*A*A*G*T*G* | SEQ ID NO. 20 | 5-10-5 |
| | C*A*mC*mA*mC*mG*mG | | |
| PA0062 | mG*mA*mC*mG*mU*G*C*A*G*A*G*G*T* | SEQ ID NO. 21 | 5-10-17 |
| | G*A*mA*mG*mC*mG*mA*mA*mG*mU*mG | | |
| | *mC*mA*mC*mA*mC*mG*mG*mU | | |
| PA0063 | mG*mA*mC*mG*mU*mG*mC*mA*mG*mA* | SEQ ID NO. 22 | 17-10-5 |
| | mG*mG*mU*mG*mA*mA*mG*C*G*A*A*G | | |
| | *T*G*C*A*C*mA*mC*mG*mG*mU | | |
| PA0064 | mG*mA*mC*mG*mU*mG*mC*mA*mG*mA* | SEQ ID NO. 23 | Full |
| | mG*mG*mU*mG*mA*mA*mG*mC*mG*mA* | | modification |
| | mA*mG*mU*mG*mC*mA*mC*mA*mC*mG* | | |
| | mG*mU | | |

A/T/G/C represented conventional unmodified deoxyribonucleotide residues; mA/mU/mG/mC represented 2′-O-methyl modified nucleotide bases; * represented phosphorothioate backbone modification.

The relative inhibition rate of the transfected antisense oligonucleotide on the expression and secretion of HBsAg by HepG2.2.1.5 was calculated according to the following formula:

Relative inhibition rate=100%*[1−(HBsAg concentration in the treatment group/HBsAg concentration in the blank group)]

The relative inhibition rates of each antisense oligonucleotide treatment group were shown in Table 6.

TABLE 6
Relative inhibition rates of oligonucleotide in Example
5 on the expression and secretion of HBsAg by HepG2.2.1.5

| | Relative inhibition rate of HBsAg |
|---|---|
| Oligonucleotide | Average value of two experiments |
| PA0054 | 61% |
| PA0020 | 60% |
| PA0063 | 57% |
| PA0061 | 55% |
| PA0043 | 54% |
| PA0056 | 54% |
| PA0053 | 53% |
| PA0055 | 53% |
| PA0052 | 52% |
| PA0044 | 51% |
| PA0058 | 51% |
| PA0047 | 51% |
| PA0059 | 51% |
| PA0051 | 50% |
| PA0057 | 50% |
| PA0045 | 49% |
| PA0041 | 49% |
| PA0062 | 48% |
| PA0049 | 48% |
| PA0042 | 46% |
| PA0046 | 46% |
| PA0048 | 45% |
| PA0060 | 41% |
| PA0064 | 36% |
| PA0050 | 35% |

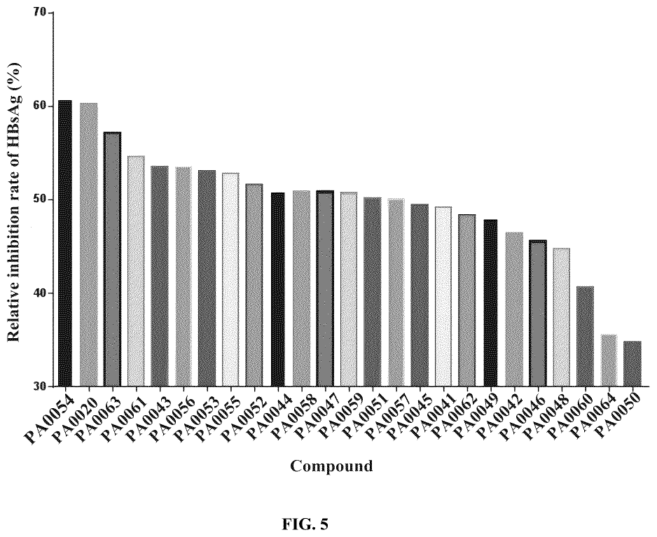

As shown in Table 6 and FIG. 5, compared with the antisense oligonucleotide PA0064 without Gap modification, the addition of Gap modification is conductive to improving the relative inhibition rate of HBsAg. By combining the results of two experiments, the weighted average of PA0020 and PA0054 was superior to antisense oligonucleotides with other Gap modification patterns.

The results of Example 5 showed that the double-Gap modification pattern of PA0020 and PA0054 is a better modification pattern.

Particularly, all CpGs in the nucleic acid sequence of PA0020 have 2′-O-methyl modifications. On the basis of PA0020, PA0020C further methylated the cytosine base of the CpG sequence, i.e., 2′-methoxy-5-methylcytosine was used to substitute cytosine to reduce its the possibility of activating Toll-like receptors (TLRs), especially TLR9, thereby reducing the risk of immune off-target toxicity.

All documents referred to herein are incorporated by reference in this application as if each is individually incorporated by reference. In addition, it should be understood that after reading the above teaching content of the present invention, those skilled in the art can make various changes or modifications to the present invention, and these equivalent forms shall also fall within the protection scope defined by the appended claims of the present application.

1. A compound, or a pharmaceutically acceptable salt, a hydrate or a solvate thereof, wherein the compound is a modified or unmodified oligonucleotide, and the oligonucleotide has a length of 24-40 nt, preferably 26-38 nt, more preferably 30-36 nt;
and the oligonucleotide has a core sequence as shown in SEQ ID NO.1:
GTGCAGAGGTGAAX1X2X3AAGTGCAC (SEQ ID NO. 1)
wherein, X₁X₂X₃ is GCG, CCG or CCT; and each T in the core sequence may be independently substituted with U;
wherein, the modification is one or more modifications selected from the following group:
(i) nucleoside modification; the nucleoside modification comprises 2′-O-methylated glycosyl modification, 2′-O-methoxyethylated glycosyl modification, and/or methylation modification of the C-5 position of cytosine; and
(ii) modification of internucleoside bonds; the modification of internucleoside bonds is that part or all of the internucleoside bonds in the oligonucleotide is substituted with a phosphorothioate internucleoside bond and/or a phosphorodithioate internucleoside bond.

2. The compound of claim 1, wherein the oligonucleotide has a structure represented by formula I:
Z1-Z2-Z3 (I)
in the formula,
Z1 is a left extension sequence located at the 5′ end of the core sequence, and the left extension sequence has a length L1 of 0-10 nt; and when L1≥1, the left extension sequence comprises nucleotides from position 11-L1 to position 10 in 5′-TCCATGCGAC-3′ successively;
Z2 is the core sequence;
Z3 is a right extension sequence located at the 3′ end of the core sequence, and the right extension sequence has a length L2 of 0-12 nt, and when L2≥1, the right extension sequence comprises nucleotides from position 1 to position L2 in 5′-ACGGTCCGGCAG-3′ successively; and
each T in the oligonucleotide may be independently substituted with U.

3. The compound of claim 1, wherein the oligonucleotide has nucleoside modifications in a region selected from the group consisting of 2-6 nts at the 5′ end, 2-3 nts in X₁X₂X₃, 2-6 nts at the 3′ end, or a combination thereof.

4. The compound of claim 3, wherein the oligonucleotide has nucleoside modifications in 2-6 nts at the 5′ end, 2-3 nts in X₁X₂X₃, and 2-6 nts at the 3′ end.

5. The compound of claim 3, wherein there is no nucleoside modification in the gap region Gap1 between the 2-6 nucleoside-modified regions at the 5′ end and X₁X₂X₃, or there is part or all of the nucleoside modifications; and/or
there is no nucleoside modification in the gap region Gap 2 between X₁X₂X₃ and 2-6 nucleoside-modified regions at the 3′ end, or there is part or all of nucleoside modifications.

6. The compound of claim 3, wherein the gap region Gap1 comprises at least L_{g1} consecutive nucleotides without nucleoside modification, wherein L_{g1} is a positive integer of 5-14, preferably 8, 9, 10, 11, 12, 13 or 14; more preferably, 10, 11, 12 or 13; and/or
the gap region Gap2 comprises at least L_{g2} consecutive nucleotides without nucleoside modification, wherein L_{g1} is a positive integer of 5-11, preferably 8-10, and more preferably 8, 9 or 10.

7. The compound of claim 1, wherein the oligonucleotide is the oligonucleotide shown in SEQ ID NO.2 or SEQ ID NO.6.

8. The compound of claim 1, wherein the compound is a modified oligonucleotide, and the compound is selected from the group consisting of:
mG*mA*mC*mG*T*G*C*A*G*A*G*G*T*G*A*A*G*mC*mG*A*A*G*T*G*C*A*C*A*mC*mG* mG*mU (PA0020); or
X₁X₂X₃mG*mA*(5Me-mC)*mG*T*G*C*A*G*A*G*G*T*G*A*A*G*(5Me -mC)*mG*A*A*G*T*G*C*A*C*A*(5Me-mC)*mG*mG*mU (PA0020C) X₁X₂X₃wherein, A, T, G, and C represent unmodified nucleoside moieties; mA, mU, mG, and mC represent nucleoside moieties modified by 2′-O-methylation; * represents a phosphorothioate bond; and (5Me-mC) represents 2-methoxy -5-methylcytidine.

9. A pharmaceutical composition, comprising the compound of any one of claim 1 or a pharmaceutically acceptable salt, a hydrate or a solvate, and a pharmaceutically acceptable carrier thereof.


SEQUENCE LISTING (25 sequences)

SEQ ID NO: 1 - DNA, 24 bp, synthetic construct
  source 1..24: mol_type = other DNA
gtgcagaggtgaasckaagtgcac

SEQ ID NO: 2 - DNA, 32 bp, synthetic construct
  source 1..32: mol_type = other DNA
  modified_base 32: mod_base = OTHER; note = uracil
gacgtgcagaggtgaagcgaagtgcacacggt

SEQ ID NO: 3 - DNA, 38 bp, synthetic construct
  source 1..38: mol_type = other DNA
acgtgcagaggtgaagcgaagtgcacacggtccggcag

SEQ ID NO: 4 - DNA, 38 bp, synthetic construct
  source 1..38: mol_type = other DNA
tccatgcgacgtgcagaggtgaagcgaagtgcacacgg

SEQ ID NO: 5 - DNA, 30 bp, synthetic construct
  source 1..30: mol_type = other DNA
acgtgcagaggtgaagcgaagtgcacacgg

SEQ ID NO: 6 - DNA, 32 bp, synthetic construct; identical to SEQ ID NO: 2
  source 1..32: mol_type = other DNA

SEQ ID NO: 7 - DNA, 36 bp, synthetic construct
  source 1..36: mol_type = other DNA
acgtgcagaggtgaagcgaagtgcacacggtccggc

SEQ ID NO: 8 - DNA, 34 bp, synthetic construct
  source 1..34: mol_type = other DNA
acgtgcagaggtgaagcgaagtgcacacggtccg

SEQ ID NO: 9 - DNA, 32 bp, synthetic construct
  source 1..32: mol_type = other DNA
acgtgcagaggtgaagcgaagtgcacacggtc

SEQ ID NO: 10 - DNA, 30 bp, synthetic construct; identical to SEQ ID NO: 5
  source 1..30: mol_type = other DNA

SEQ ID NO: 11 - DNA, 25 bp, synthetic construct
  source 1..25: mol_type = other DNA
acgtgcagaggtgaagcgaagtgca

SEQ ID NO: 12 - DNA, 36 bp, synthetic construct
  source 1..36: mol_type = other DNA
catgcgacgtgcagaggtgaagcgaagtgcacacgg

SEQ ID NO: 13 - DNA, 34 bp, synthetic construct
  source 1..34: mol_type = other DNA
tgcgacgtgcagaggtgaagcgaagtgcacacgg

SEQ ID NO: 14 - DNA, 32 bp, synthetic construct
  source 1..32: mol_type = other DNA
cgacgtgcagaggtgaagcgaagtgcacacgg

SEQ ID NO: 15 - DNA, 25 bp, synthetic construct
  source 1..25: mol_type = other DNA
cagaggtgaagcgaagtgcacacgg

SEQ ID NO: 16 - DNA, 40 bp, synthetic construct
  source 1..40: mol_type = other DNA
acacacacacacacacacacacacacacacacacacacac

SEQ ID NO: 17 - DNA, 32 bp, synthetic construct; identical to SEQ ID NO: 2
  source 1..32: mol_type = other DNA
  modified_base order(5,32): mod_base = OTHER; note = uracil

SEQ ID NO: 18 - DNA, 20 bp, synthetic construct
  source 1..20: mol_type = other DNA
  modified_base 5: mod_base = OTHER; note = uracil
gacgtgcagaggtgaagcga

SEQ ID NO: 19 - DNA, 20 bp, synthetic construct
  source 1..20: mol_type = other DNA
  modified_base order(1,20): mod_base = OTHER; note = uracil
tgaagcgaagtgcacacggt

SEQ ID NO: 20 - DNA, 20 bp, synthetic construct
  source 1..20: mol_type = other DNA
  modified_base 2: mod_base = OTHER; note = uracil
gtgaagcgaagtgcacacgg

SEQ ID NO: 21 - DNA, 32 bp, synthetic construct; identical to SEQ ID NO: 2
  source 1..32: mol_type = other DNA
  modified_base order(5,23,32): mod_base = OTHER; note = uracil

SEQ ID NO: 22 - DNA, 32 bp, synthetic construct; identical to SEQ ID NO: 2
  source 1..32: mol_type = other DNA
  modified_base order(5,13,32): mod_base = OTHER; note = uracil

SEQ ID NO: 23 - DNA, 32 bp, synthetic construct; identical to SEQ ID NO: 2
  source 1..32: mol_type = other DNA
  modified_base order(5,13,23,32): mod_base = OTHER; note = uracil

SEQ ID NO: 24 - DNA, 3182 bp, synthetic construct
  source 1..3182: mol_type = other DNA
ttccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaa

SEQ ID NO: 25 - DNA, 46 bp, synthetic construct
  source 1..46: mol_type = other DNA
ctgccggaccgtgtgcacttcgcttcacctctgcacgtcgcatgga